(19) United States
(12) Patent Application Publication
(10) Pub. No.: US 2003/0228265 A1
(43) Pub. Date: Dec. 11, 2003

(54) Herbal composition and uses thereof

(76) Inventor: Phil Lewis Rasmussen, Auckland (NZ)

Correspondence Address: JACOBSON HOLMAN PLLC, 400 SEVENTH STREET N.W., SUITE 600, WASHINGTON, DC, 20004, US

(21) Appl. No.: 10/424,136

(22) Filed: Apr. 28, 2003

(30) Foreign Application Priority Data
Apr. 26, 2002 (NZ) 518044

(51) Int. Cl. A61K 7/26; A61K 35/78; A61K 31/70

(52) U.S. Cl. 424/58; 424/725; 514/23; 424/757; 424/745; 424/747

(57) ABSTRACT

A herbal composition useful in the treatment and/or prophylaxis of oral pathogens comprising a composition having either an effective amount of Manuka extract, or Tanekaha extract or a mixture of both Manuka and Tanekaha.

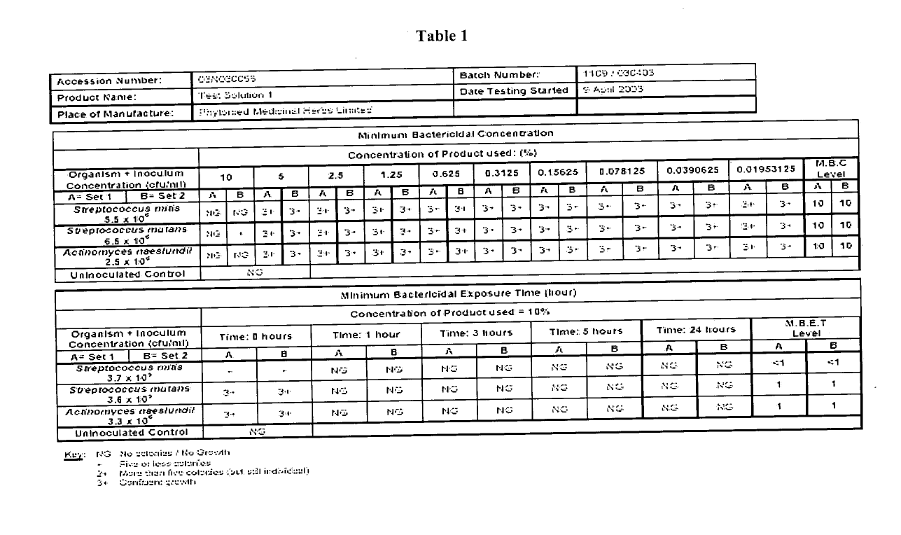

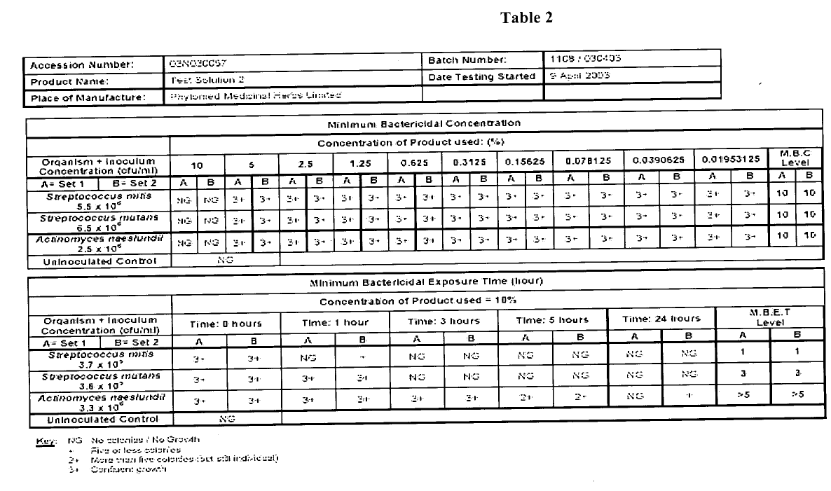

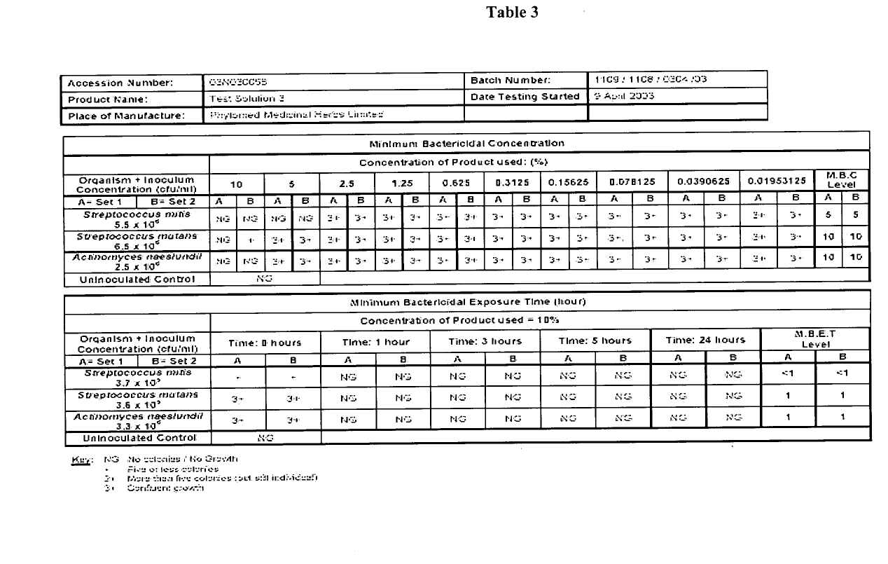

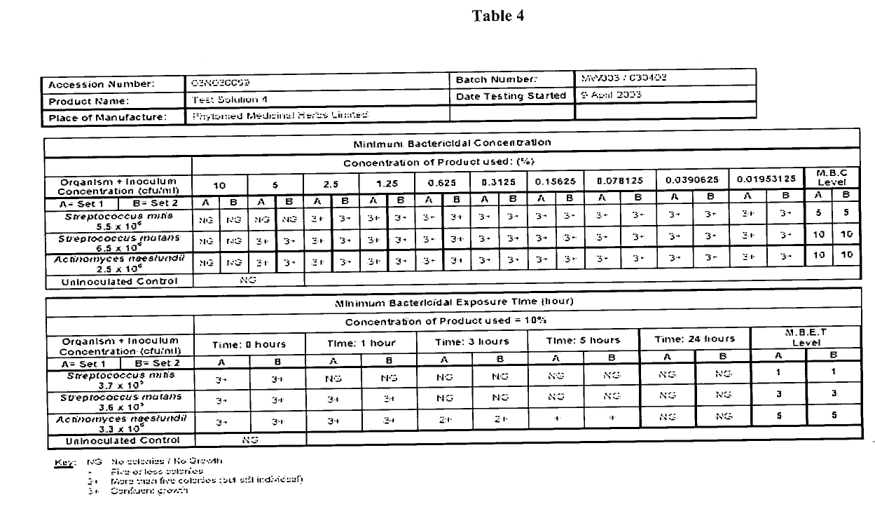

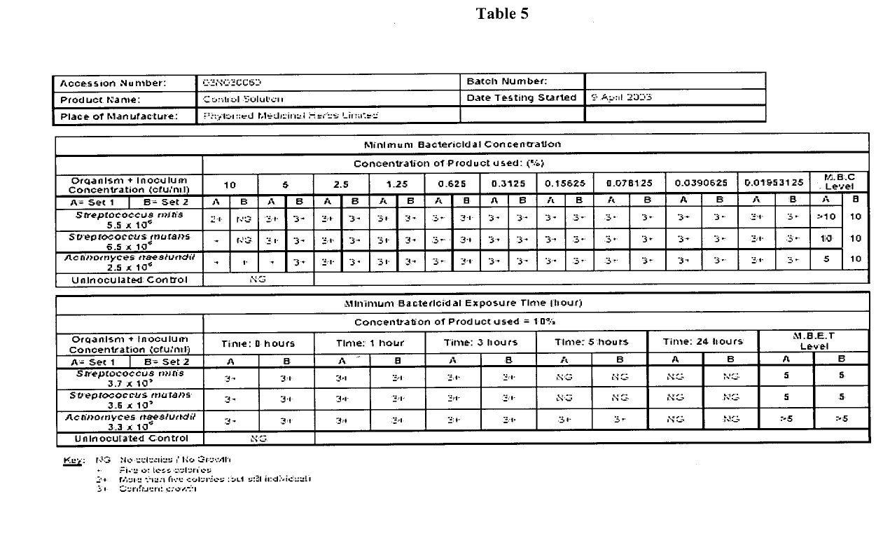

### FIELD OF THE INVENTION

The present invention relates to a herbal composition useful as a method of treatment and/or prophylaxis of oral pathogens.

### BACKGROUND TO THE INVENTION

People suffering from oral disease caused by oral pathogens, such as gum disease dental caries and/or halitosis, find such diseases a social inhibitor, such that it affects a person's social interaction and loss of dignity. In some cases affected people do not realise they suffer from gum disease and left untreated can lead to serious loss of gum and eventual loss of teeth.

Such pathogens are in hard to reach places inside the mouth, where conventional cleaning methods of brushing the teeth or flossing are often not sufficient by themselves to remove the pathogens causing halitosis and/or gum disease. Indeed such pathogens can be harboured in pieces of food remaining in the mouth and if the pathogens are provided with an environment to their liking, such pathogens can get under the gum, thus brushing with a tooth brush will not remove such pathogens.

Conventional mouthwashes used for the treatment and/or prophylaxis of these conditions, such as those based upon chlorhexidine, have certain disadvantages. These can include their unpleasant taste, staining of the teeth with prolonged use, a burning sensation of the tongue on initial use, and altering taste sensation. These factors can make it very difficult to encourage young children (who have high caries risk) to use chlorhexidine. Antibiotic and chlorhexidine resistant strains of oral pathogens, have also emerged in recent years.

In terms of herbal remedies, those containing tannins such as manuka (Leptospermuni scoparium) and tanekaha (Phyllocladus trichomanoides) can be useful in attacking oral pathogens as some tannins and related phytochemicals have such activity.

### OBJECT OF THE INVENTION

It is an object of the present invention to provide a herbal composition which has advantages over what is currently known or which at least provides the public with a useful choice.

### SUMMARY OF THE INVENTION

In a first aspect of the invention there is provided a herbal composition useful in the treatment and/or prophylaxis of oral pathogens comprising or including an effective amount of Manuka extract.

Preferably the manuka extract is a hydroethanolic extract.

Preferably the composition includes one or more of the following:

liquid carriers(s),

excipients(s),

compatible carrier(s),

orally acceptable carrier(s) and/or, flavouring agent(s).

Preferably the excipients and/or flavouring agents and/or carriers (which may take the form of a hydroethanolic extract and/or essential oil) may include:

Liquorice liquid extract,

Aniseed liquid extract,

Peppermint essential Oil,

Mint, and/or

Honey.

Preferably also benzoic acid (as a preservative) and/or glycerine may be added.

Preferably the composition is administered as a mouthwash. Alternatively the composition is administered as an oral spray.

Preferably the manuka extract is sourced from the East Cape of New Zealand.

Preferably the effective amount of manuka extract is between 10 to 50% v/v.

Preferably the effective amount is between 10 to 30% v/v.

Preferably the liquid carrier is de-ionised water and/or ethanol.

In a second aspect of the invention there is provided a herbal composition useful in the treatment and/or prophylaxis of oral pathogens comprising or including an effective amount of Tanekaha extract.

Preferably the Tanekaha extract is a hydroethanolic extract.

Preferably the composition includes one or more of the following:

liquid carrier(s),

excipient(s),

compatible carrier(s),

orally acceptable carriers, and/or

flavouring agents.

Preferably the excipients and/or flavouring agents and/or carriers (which may take the form of a hydroethanolic extract and/or essential oil) may include:

Liquorice liquid extract,

Aniseed liquid extract,

Peppermint essential Oil,

Mint, and/or

Honey.

Preferably also benzoic acid (as a preservative) and/or glycerine may be added.

Preferably the composition is administered as a mouthwash. Alternatively the composition is administered as an oral spray.

Preferably the Tanekaha is sourced from the trees found in their natural habitat in the New Zealand bush.

Preferably the effective amount of Tanekaha extract is between 5 to 50% v/v.

Preferably the liquid carrier is de-ionised water and/or ethanol.

In another aspect of the present invention there is provided a herbal composition useful in the treatment and/or prophylaxis of oral diseases comprising or including:

an effective amount of manuka extract, and/or

an effective amount of tanekaha extract.

Preferably one or both of the manuka and tanekaha extracts are hydroethanolic extracts.

Preferably the composition is a synergistic composition.

Preferably there is further included an effective amount of manuka essential oil Preferably the composition includes one or more of the following:

liquid carrier(s),

excipient(s),

compatible carrier(s),

orally acceptable carrier(s), and/or flavouring agent(s).

Preferably the excipients and/or flavouring agents and/or carriers (which many take the form of a hydroethanolic extract and/or essential oil) may include any one or more of the following:

Liquorice liquid extract,

Aniseed liquid extract,

Peppermint essential Oil,

Mint, and/or

Honey.

Preferably also benzoic acid (as a preservative) and/or glycerine may be added.

Preferably the composition is administered as a mouthwash. Alternatively the composition is administered as an oral spray.

Preferably the manuka extract is sourced or derived from the East Cape of New Zealand.

Preferably the Tanekaha is sourced from the trees found in their natural habitat in the New Zealand bush.

Preferably the effective amount of manuka extract is between 10 to 50% v/v.

Preferably the effective amount of manuka extract is between 20 to 30% v/v.

Preferably the effective amount of tanekaha extract is between 5 to 50% v/v.

Preferably the effective amount of tanekaha extract is between 10 to 30% v/v.

Preferably the liquid carrier is de-ionised water and/or ethanol.

Preferably the Manuka extract and oil can be sourced or derived from Tairawhiti Pharmaceuticals who produce this from Manuka harvested from the East Cape of New Zealand.

Preferably the Tanekaha and Manuka liquid extracts are extracted from the raw material using a cold percolation extract or a maceration process.

In a further aspect of the invention there is provided a method of extracting manuka and/or tanekaha to form one or more hydroethanolic extracts for use in a herbal composition useful in the treatment and/or prophylaxis of oral pathogens comprising or including the steps of:

Preferably the combination of the liquid extracts obtained from 6) and/or 7) produces the finished plant extract.

Alternatively and/or additionally, extracts may be obtained by simply blending/homogenising fresh herb with a mixture of ethanol and water, leaving to macerate for two weeks, then straining and pressing.

Preferably the extracts of manuka and/or tanekaha are combined with one or more of the following:

liquid carriers,

more excipient(s),

one or more compatible carriers, rally acceptable carrier(s), and/or

flavouring agent(s).

Preferably the excipients and/or flavouring agents and/or carriers (which many take the form of a hydroethanolic extract and/or essential oil) may include any one or more of the following:

Liquorice liquid extract,

liquid extract,

Peppermint essential Oil,

Mint, and/or

Honey.

Preferably also benzoic acid (as a preservative) and/or glycerine may be added.

In a further aspect of the invention there is provided one or both of manuka and/or tanekaha extracts as hydroethanolic extracts for use in a herbal composition useful in the treatment and/or prophylaxis of oral pathogens and/or oral diseases prepared or extracted substantially according to the above method.

In a further aspect the present invention consists in the use of such a herbal composition for the treatment and/or prophylaxis of oral diseases.

Preferably the oral diseases include halitosis and/or gum disease. The elimination from the mouth of food material which can be difficult to remove through methods of brushing or flossing alone, is an additional application of the present invention.

As used herein the term “and/or” means “and” or “or” and a noun followed by “(s)” means the singular or plural

### BRIEF DESCRIPTION OF THE TABLES

Table 1 details the results of the testing made for a solution containing manuka only,

Table 2 details the results of the testing made for a solution containing tanekaha only,

Table 3 details the results of the testing made for a solution containing manuka and tanekaha, and

Table 4 details the results of the testing mode with the mouthwash containing manuka and tanekaha.

Table 5 details the results of the testing made using the control solution.

### DETAILED DESCRIPTION OF THE INVENTION

The present invention has surprisingly shown that the use of the traditional New Zealand herbs Manuka (Leplospermum scoparium) and Tanekaha (Phyllocladus trichomanoides) administered either together or separately as a mouthwash provides an effective treatment against such oral diseases, especially the oral diseases of halitosis and gum disease.

The Manuka and Tanekaha have been sourced from New Zealand Native trees using an extraction process to obtain the herbs as hydroethanolic extracts. These extracts have been found to exhibit special characteristics including in vitro bactericidal activity against 3 known oral pathogens, Streptococcus mitis, Streptococcus mutans, and Actinomyces naeslundii.

The hydroethanolic extraction processes used (in the manufacture of these extracts), have been found to extract not only tannins, but also a range of other phytochemicals including terpenes and volatile oils, which contribute to this activity against oral pathogens.

The activity of a herbal composition of the present invention has been found to prevent and or treat halitosis and/or other infections causing mouth diseases.

This has been shown by the following experiment where the bacterial pathogens studied were Streptococcus mutan; Streptococcus mitis; and Actinomyces naeslundii. These bacterium are recognised as the species most implicated as oral pathogens contributory to dentals problems as reported by the Microbiology Unit at Otago University Dental School and are plaque-forming gram positive bacteria found in the mouth known to contribute to the development of the oral diseases of dental caries, halitosis and/or gum disease.

The composition of the invention can be used in any number of ways as would be known in the art. However, specifically the invention is intended to be shaken before use, then 2-4 ml diluted with 20-30 ml of water, and used to rinse the mouth for a period of 20-30 seconds, twice daily. The liquid should then be expelled from the mouth.

One preferred method of formulating the composition is described below, but is not limiting. To prepare 100 millilitres (mls) of the composition, this is made from the following ingredients:

Manuka 1 in 2 strength hydroethanolic extract: 24.5 mls

Tanekaha 1 in 2 strength hydroethanolic extract: 17.5 mls

Aniseed 1 in 2 strength hydroethanolic extract: 7.0 mls

Liquorice 1 in 1 strength hydroethanolic extract: 13.0 mls

Manuka steam distilled volatile oil: 0.5 mls

Peppermint steam distilled volatile oil: 0.5 mls, and

Purified Water: 37.1 mls.

The term “1 in 2 strength” means that for every 2 mls of volume of extract, this is equivalent to 1 gram of the actual herbal starting material.

The composition is formed by first mixing the individual hydroethanolic liquid extracts with the essential oils, and then adding in purified water. Mixing is conducted at room temperature, in a controlled pharmaceutical manufacturing environment. A controlled pharmaceutical manufacturing environment includes an environment where the air has firstly been cleaned and then pressurised, the people preparing the composition wear gloves, gowns and over shoes and all preparation equipment has been cleaned or wiped down with an aseptic technique. The extracts can be obtained from a procedure according to the following:

1 Powdering dried manuka and/or tanekaha (hereafter “plant”),

2 Wetting the dried plant from above with a mixture of ethanol and water,

3 Packing the wetted plant material into a percolator vessel,

4 Addition of a mixture of more ethanol and water to the plant material in the percolator vessel,

5 Allowing the ethanol/water mixture to slowly permeate down the length of the percolator vessel, extracting active constituents from the plant in the process,

6 Collecting the plant extract thus produced at the outlet tap of the percolator.

Preferably the method further includes the step:

7 Once percolation is complete, pressing the remaining plant material to extract remaining hydroethanolic extract.

This composition is then diluted in water to the strength required and used.

Patients who had the bacterial pathogens (ie Streptococcus mutans; Streptococcus mitis; and Actinomyces naeslundii) were then administered the diluted composition into the mouth. Each patient rinsed their mouth with the composition for 20-30 seconds and then expelled the composition. Initially the user experience a refreshing and cleansing sensation that tasted of peppermint and manuka. This was followed by a mildly astringent or drying sensation in the mouth. In addition, during rinsing of the mouth followed by expelling of the diluted composition, users reported the removal of previously retained food material from the mouth. It is thought that the tannins present in the composition aid in the dislodging of the food material from the mouth.

The users of the composition discovered that the use of the composition aided in the treatment of the ailments associated with the bacterial pathogens described above, eg halitosis, gum disease etc.

Experimental Data

The composition has been used in the following experiment to determine the effects of the composition.

The experiment was carried out using four herbal liquid composition. These composition were:

i) manuka only

ii) tanekaha only

iii) manuka and tanekaha and

iv) a mouthwash formulation containing manuka and tanekaha in 25% ethanol.

The extracts were tested in vitro against the specified bacteria of Streptococcus mutans; Streptococcus mitis; and Actinomyces naeslundii to determine the effect the four compositions would have on the survival rate of the bacteria. The extracts tested were prepared according to the method described above. The methodology used was according to Disinfection, Sterilisation, and Preservation (4^{th} Edition), Chapter 58. Two modifications were made to the method, 1) the incubation conditions were altered to accommodate the type of organisms being used, and 2) the plating of the test samples at differing periods was made to determine the time required for the composition to kill the test organisms.

Test Organisms

The following organisms were used in the testing. Cultures were maintained in accordance with the recommendations of the Curator of the Culture Collections Centre NZ Communicable Disease Centre.

| | |
|---|---|
| Streptococcus mitis | NZRM¹ 2989 |
| Streptococcus mutans | NZRM 987 |
| Actinomyces naeslundii | NZRM 1004 |

Methodology

The herbal liquid extracts and control (containing 25% ethanol) were tested. The solutions that were tested as follows.

| Solution | Composition of the Solution |
|---|---|
| 1 | Manuka 1 in 2 strength hydroethanolic extract diluted to 24.5% |
| 2 | Tanekaha 1 in 2 strength hydroethanolic extract diluted to 17.5% |
| 3 | Manuka 1 in 2 strength hydroethanolic extract diluted to 24.5% |
| 4 | Mouthwash preparation containing manuka extract 1 in 2 |
| | strength hydroethanolic extract (24.5%), tanekaha 1 in 2 |
| | strength hydroethanolic extract (17.5%), manuka essential |
| | oil 0.5%, liquorice 1 in 1 strength hydroethanolic extract |
| | 13%, aniseed 1 in 2 strength hydroethanolic extract 7%, |
| | and peppermint essential oil 0.5% |
| 5 | Control ethanol 25% solution. |

¹NZRM: New Zealand Reference Culture Collection - Medical Section

Minimal Bactericidal Concentration (MBC). This was carried out by making a series of twofold dilutions of the test antimicrobial agent starting at 10% (which is usage concentration) in a culture medium (Brain Heart Infusion Broth). These dilutions were then used as the samples for testing. Ten mls of each dilution was inoculated with 0.1 ml of an overnight culture (10⁶-10⁸ cfu/ml) and mixed. All dilutions were then incubated anaerobically at 37° C. for 19 hours.

To determine the concentration of antimicrobial agent that either fails to show bgrowth or results in a 99.9% decrease, each dilution was subcultured onto a medium free of antimicrobial agents and incubated. A volume of 0.02 mls of each of the dilutions was subcultured on Tryptic Soy Agar (TSA) and incubated under anaerobic conditions at 37° C. for 48 hours. All testing was performed in duplicate as two sets A and B.

All enumeration of each organism used was performed to obtain the starting inoculum levels.

For enumeration of all organisms, TSA was incubated at 37° C. anaerobically for used.

Each test included a positive control (Brain Heart Infusion Broth plus test organism), a negative control (Brain Heart Infusion Broth only) and a solution of the test sample only. Each test sample was also tested for the presence of any naturally occurring anaerobic bacterial that may interfere with the testing regime.

Minimum Bacterial Exposure Time (MBET)

The MBC test showed that a 10% concentration for each product under test was necessary to either completely kill Streptococcus mutans or to decrease its growth by 99.9%. This percentage was then used for further testing.

A 10% solution of each product under test was prepared on the basis of the MBC test and then used to inoculate each of the test organisms. Each preparation was then immediately subcultured onto TSA plates to give an initial zero time reading and then incubated under anaerobic conditions at 37° C. This testing was carried out in duplicate for a period of 48 hours.

At the time intervals of 1, 3, 5 and 24 hours each subculture was checked for the number of colonies present.

Results

Results for each of the 4 herbal and 1 control solutions tested are presented in the tables below.

Growth of Streptococcus mitis was significantly reduced by both the manuka (MBET<1 hr) and tanekaha (MBET 1 hr) extracts, with a Minium Bactericidal Concentration (MBC) fore each diluted hydroethanolic extract of 10% being required to kill this organism. A synergistic bactericidal activity was revealed for a combination of manuka and tanekaha (test solution 3), with MBC of only 5% being required to kill streptococcus mitis (MBET<1 hr). The finished mouthwash preparation (test solution 4) containing the same concentrations of manuka and tanekaha extracts and smaller amounts of other ingredients, also had an MBC level of 5% (MBET 1 hr).

Growth of Streptococcus mutans was also reduced by both manuka (MBC 10%; MBET 1 hr) and tanekaha (MBC 10%; MBET 3 hrs) individual diluted extracts, with similar levels of antimicrobial activity being measured for the combined manuka and tanekaha extracts (MBC 10%; MBEC 1 hr) and finished mouthwash formulation (MBC 10%;MBEC 3 hrs).

Growth of Actinomyces naeslundii was reduced by both manuka (MBC 10%; MBET 1 hr) and tanekaha (MBC 10%; MBET>5 hr) individual diluted extracts, with again similar levels of antimicrobial activity being observed for both the combined manuka and tanekaha extracts (MBC 10%; MBET 1 hr) and finished mouthwash formulation (MBC 10%; MBET 5 hrs).

Discussion

Inhibition of the growth of 3 oral pathogenic species of bacteria, Streptococcus mutans; Streptococcus mitis, and Actinomyces naeslundii, was produced by diluted hydroethanolic extracts of manuka, tanekaha, as well as a combination of manuka and tanekaha, and a mouthwash formulations containing this combination with small amounts of other ingredients. This activity was particularly pronounced against Streptococcus mitis, but was also evident against the other 2 bacterial species. High levels of each of these 3 species of bacteria, have been associated with the development of caries in the oral cavity, and poor oral health.

### REFS

Tinanoff N et al, Pediatr Dent 24(6):543-51, 2002.

Kamma J J et al, Oral Microbiol Immunol 15(2):103-11, 2000.

Feres M et al, J Clin Periodontol 26(12):784-92, 1999.

Bowden G H, Community Dent Oral Epidemiol 25(1):76-81, 1997.

Anderson M, Pediatr Dent 24(5):377-85, 2002.

What we claim is:

1. A herbal composition useful in the treatment and/or prophylaxis of oral pathogens comprising or including an effective amount of Manuka extract.

2. A composition as claimed in claim 1 wherein said Manuka extract is a hydroethanolic extract and/or an essential oil.

3. A composition as claimed in claim 2 wherein said composition includes one or more of the following:
liquid carrier(s),
excipient(s),
compatible carrier(s),
acceptable carrier(s), and
flavouring agent(s).

4. A composition as claimed in claim 3 wherein said liquid carrier is de-ionised water and/or ethanol.

5. A composition as claimed in claim 3 wherein said excipient(s), flavouring agent(s) and/or carrier(s) may include any one or more of the following:
Liquorice liquid extract,
Aniseed liquid extract,
Peppermint essential Oil,
Mint, and/or
Honey.

6. A composition as claimed in any one of claims 1 to 5 wherein said composition may further include benzoic acid and/or glycerine.

7. A composition as claimed in any one of claims 1 to 6 wherein said composition is administered as a mouthwash or as an oral spray.

8. A composition as claimed in any one of claims 1 to 7 wherein said Manuka extract is sourced from the East Cape of New Zealand.

9. A composition as claimed in any one of claims 1 to 8 wherein said effective amount of Manuka extract is between 10 and 50% v/v.

10. A composition as claimed in claim 9 wherein said effective amount of manuka extract is between 20 to 30% v/v.

11. A herbal composition useful in the treatment and/or prophylaxis of oral pathogens comprising or including an effective amount of Tanekaha extract.

12. A composition as claimed in claim 11 wherein said Tanekaha extract is a hydroethanolic extract and/or essential oil.

13. A composition as claimed in any one of claims 11 to 12 wherein said composition includes one or more of the following:
liquid carrier(s),
excipient(s),
compatible carrier(s)
orally acceptable carrier(s), and/or
flavouring agent(s).

14. A composition as claimed in claim 13 wherein said liquid carrier is de-ionised water and/or ethanol.

15. A composition as claimed in claim 13 wherein said excipient(s), flavouring agent(s) and/or carrier(s) may include any one or more of the following:
Liquorice liquid extract,
Aniseed liquid extract,
Peppermint essential Oil,
Mint, and/or
Honey.

16. A composition as claimed in any one of claims 11 to 15 wherein said composition may further include benzoic acid and/or glycerine.

17. A composition as claimed in any one of claims 11 to 16 wherein said composition is administered as a mouthwash or as an oral spray.

18. A composition as claimed in any one of claims 11 to 17 wherein said Tanekaha extract is sourced from the trees found in their natural habitat in the New Zealand bush.

19. A composition as claimed in any one of claims 10 to 18 wherein said effective amount of Tanekaha extract is between 5 to 50% v/v.

20. A composition as claimed in claim 19 wherein said effective amount of Tanekaha extract is between 10 to 25% v/v.

21. A herbal composition useful in the treatment and/or prophylaxis of oral diseases and/or pathogens comprising or including:
an effective amount of Manuka extract, and
an effective amount of Tanekaha extract.

22. A composition as claimed in claim 21 wherein said one or both of said Manuka and Tanekaha extracts are hydroethanolic extracts and/or essential oils.

23. A composition as claimed in claims 21 or 22 wherein said composition is a synergistic composition.

24. A composition as claimed in any one of claims 21 to 23 wherein said composition includes any one or more of the following:
liquid carrier(s),
excipient(s),
compatible carrier(s),
orally acceptable carrier(s),
flavouring agent(s).

25. A composition as claimed in any one of claims 21 to 23 wherein said excipient(s), flavouring agents and/or carrier(s) may include any one or more of the following:
Liquorice liquid extract,
Aniseed liquid extract,
Peppermint essential Oil,
Mint, or
Honey.

26. A composition as claimed in claim 24 wherein said liquid carrier is de-ionised water and/or ethanol.

27. A composition as claimed in any one of claims 21 to 26 wherein said composition may further include benzoic acid or glycerine.

28. A composition as claimed in any one of claims 21 to 27 wherein said composition is administered as a mouthwash or as an oral spray.

29. A composition as claimed in any one of claims 21 to 28 wherein said Manuka extract is sourced or derived from the East Cape of New Zealand and said Tanekaha extract is sourced from the trees found in their natural habitat in the New Zealand bush.

30. A composition as claimed in any one of claims 21 to 29 wherein said effective amount of Manuka extract is between 10 to 50% v/v and said effective amount of Tanekaha extract is between 5 to 50% v/v.

31. A composition as claimed in claim 30 wherein said effective amount of Manuka extract is between 20 to 30% v/v and said effective amount of Tankekaha extract is between 10 to 25% v/v.

32. A composition as claimed in any one of the preceding claims wherein said Tanekaha and Manuka liquid extracts are extracted from the raw material using a cold percolation extract or a maceration process.

33. A composition of extracting Manuka and/or tanekaha to form one or more hydroethanolic extracts for use in a herbal composition useful in the treatment and/or prophylaxis of oral diseases and/or pathogens comprising or including the steps of:
1 Powdering dried Manuka and/or Tanekaha (hereafter “plant”),
2 Wetting the dried plant from above with a mixture of ethanol and water,
3 Packing the wetted plant material into a percolator vessel,
4 Addition of a mixture of more ethanol and water to the plant material in the percolator vessel,
5 Allowing the ethanol/water mixture to slowly permeate down the length of the percolator vessel, extracting active constituents from the plant in the process,
6 Collecting the plant extract thus produced at the outlet tap of the percolator.

34. A composition as claimed in claim 33 wherein said composition further includes the step:
7 Once percolation is complete, pressing the remaining plant material to extract remaining hydroethanolic extract.

35. A composition as claimed in any one of claims 1 to 30 wherein said Manuka and/or Tankekaha extracts are obtained from a method as defined in any of claims 32 to 33.

36. Use of a composition as claimed in any one of claims 1 to 30 for the treatment and/or prophylaxis of oral diseases.

37. The use of a composition as claimed in claim 33 wherein said oral diseases and/or includes halitosis and/or gum diseases and/or pathogens.
